(19) United States
(12) Patent Application Publication
(10) Pub. No.: US 2004/0073257 A1
(43) Pub. Date: Apr. 15, 2004

(54) Methods and apparatus for the repair of hernias

(76) Inventor: Gregory A. Spitz, Aurora, IL (US)

Correspondence Address: FRANK C. NICHOLAS, CARDINAL LAW GROUP, Suite 2000, 1603 Orrington Avenue, Evanston, IL, 60201, US

(21) Appl. No.: 10/267,188

(22) Filed: Oct. 9, 2002

(51) Int. Cl. A61B 17/08

(52) U.S. Cl. 606/220

(57) ABSTRACT

The present invention relates to devices and methods for the repair of hernia. One method for repairing a hernia the steps of making an incision through a skin layer of a patient near the hernia, creating an entrance into the preperitoneal space above the peritoneum at a location above the hernia, identifying and freeing a hernia sac, creating a pocket in the preperitoneal space, directing a surgical patch down through the incision and into the preperitoneal space, and expanding the surgical patch in the preperitoneal space. The method further includes the steps of inserting a distal end of a surgical fastening device through the incision and into the surgical patch, actuating the surgical fastening device to drive a fastener through the surgical patch and into the tissue of the patient, moving the distal end of the surgical fastening device to another location, actuating the surgical fastening device to drive a second fastener through the surgical patch and into the tissue of the patient, and closing the incision with stitches. One surgical apparatus for repairing a hernia includes a surgical stapling instrument for applying at least one surgical staple to fasten a surgical hernia patch to internal body tissue. The surgical instrument includes a handle assembly have a longitudinal axis. A staple cartridge housing is mounted to the handle assembly and is adapted to receive the at least one staple. The staple cartridge housing is dimensioned for insertion through an incision and has a staple actuator mechanism for applying the at least one staple into tissue. An actuation mechanism is operatively coupled to the staple actuator mechanism to operate the staple actuation mechanism.

### TECHNICAL FIELD

The present invention generally relates to the repair of internal body tissue. More particularly, the present invention relates to surgical instruments and procedures that can be used for the repair of hernias.

### BACKGROUND OF THE INVENTION

A hernia is a defect in a muscle of a person through which internal body organs can protrude into the inguinal tissue. This can happen in the groin area, the abdominal wall, the bowels, the diaphram, the scrotal sac or even a disk in the vertebral bones. Hernias can cause discomfort as well as a lump under the skin. The most common type of hernia occurs in the abdomen, in which part of the intestines protrude through the abdominal wall to form a hernial sac. When such a hernia occurs in the abdominal region, conventional corrective surgery has been required to correct the defect.

Surgical mesh materials or patches have been developed for the repair of hernias. These mesh materials help reinforce and close the hernia. Various surgical techniques have been utilized to apply and secure the surgical mesh over the hernia. In one surgical approach, laparascopy techniques and devices are utilized to apply a surgical mesh from a remote location under the hernia to be repaired. This surgical operation generally involves repairing the hernia by retracting the intra-abdominal contents away from the hernia defect and then inserting a bundle of surgical mesh into the patient to block the defect. A surgical patch is usually secured over the mesh to hold it in place.

However, surgical operations utilizing such laparacopic devices and techniques can be complicated. In addition, such operations typically require the use of general anesthesia and costly disposable instrumentation to support the laparoscopic surgery. In addition, this surgical technique can suffer from the difficulty of spreading and holding the surgical mesh over the defect in a satisfactory manner. Further, it may be difficult to affix the surgical patch in a smoothly expanded manner without causing substantial subsequent tension on the abdominal portions to which the mesh is affixed.

In another surgical approach, a hernia can be repaired by attaching a surgical patch directly over the hernia. In this technique, a surgeon opens the abdominal cavity of a patient by a surgical incision through the major abdominal muscles. Several layers of the abdominal wall are generally separated to reach the herniated portions and to prepare an opening for the insertion of the surgical patch. Before the surgical patch is inserted into the patient, 4-12 sutures are passed under a memory recoil ring located near the perimeter of the patch. The surgeon then folds and compacts the surgical patch and inserts the patch through the incision into the patient's preperitoneal space. Thereafter, the surgeon uses his fingers to move and flatten out the patch within the preperitoneal space to ensure that none of the edges of the patch are flipped back. Once the hernia mesh patch covers the defect in the patient's abdominal cavity, the edges of the fascial defect are lifted and the perimeter sutures previously placed through the patch are passed through the peritoneum and posterior fascia/sheath. The sutures are then tied and trimmed.

However, if the sutures are not passed through the tissue directly above the ring of the patch, the sutures may be placed under tension and the patch may become distorted. In addition, it can also be time consuming for the surgeon to secure all of the sutures in place. Further, the surgical patch utilized in this technique typically includes the use of a resilient circumferential ring located near the outer edge that creates tension throughout the patch to help expand the patch, thereby increasing the cost of the patch.

### SUMMARY OF THE INVENTION

In view of the above, the present invention provides surgical apparatus and procedures for the repair of hernia. The surgical apparatus and procedures provide a low cost and efficient procedure for the repair hernias. The surgical apparatus and procedures also allow a hernia to be repaired with less tension, a smaller wound or incision, superior fixation and at a potentially lower cost to the patient than traditional methods. The apparatus and procedures further allow a surgeon to secure a patch over a defect without the use of sutures and in less time than traditional surgical procedures.

One method for repairing a hernia in accordance with the present invention includes the steps of making an incision through a skin layer of a patient near the hernia, creating an entrance into the preperitoneal space above the peritoneum at a location above the hernia, identifying and freeing a hernia sac, creating a pocket in the preperitoneal space, directing a surgical patch down through the incision and into the preperitoneal space, and expanding the surgical patch in the preperitoneal space. The method further includes the steps of inserting a distal end of a surgical fastening device through the incision and into the surgical patch, actuating the surgical fastening device to drive a fastener through the surgical patch and into the tissue of the patient, moving the distal end of the surgical fastening device to another location, actuating the surgical fastening device to drive a second fastener through the surgical patch and into the tissue of the patient, and closing the incision with stitches.

One surgical apparatus in accordance with the present invention includes a surgical patch applicator for positioning a patch of surgical patch over a hernia defect. The surgical patch applicator generally includes an elongated member having a first end and a second end. The second end is sized for insertion into an opening of the surgical hernia patch. A lumen extends through the elongated member and a balloon is coupled to the second end of the elongated member. The balloon inflates in a planar direction to expand the surgical hernia patch over the hernia defect when a fluid is introduced into the lumen.

Another surgical apparatus in accordance with the present invention includes a surgical stapling instrument for applying at least one surgical staple to fasten a surgical hernia patch to internal body tissue. The surgical instrument includes a handle assembly have a longitudinal axis. A staple cartridge housing is mounted to the handle assembly and is adapted to receive the at least one staple. The staple cartridge housing is dimensioned for insertion through an incision and has a staple actuator mechanism for applying the at least one staple into the tissue. An actuation mechanism is operatively coupled to the staple actuator mechanism to operate the staple actuation mechanism. A surgeon can rotate the staple cartridge housing in order to apply staples at desired surgical locations.

The invention, together with further attendant advantages, will best be understood by reference to the following detailed description of the presently preferred embodiments of the invention, taken in conjunction with the accompanying drawings. It is to be understood that both the foregoing general description and the following detailed description are exemplary and explanatory and are intended to provide further explanation of the invention as claimed.

### BRIEF DESCRIPTION OF THE DRAWINGS

A preferred embodiment of the present invention will be described in detail below in connection with the drawings in which:

FIG. 1 is a diagrammatical view of the repair of a hernia of a patient using surgical apparatus in accordance with the present invention;

FIG. 2 is a fragmentary diagrammatical cross-sectional view of an incision in an abdominal wall of a patient having a hernia, wherein a surgical hernia patch is being prepared for insertion into the incision of the patient;

FIG. 3 is a fragmentary diagrammatical cross-sectional view of the incision in the abdominal wall of FIG. 2, wherein the surgical hernia patch is being inserted into hernia of the patient;

FIG. 4 is a fragmentary diagrammatical cross-sectional view of the incision in the abdominal wall of FIG. 2, wherein a balloon of a surgical patch applicator is inserted into the incision and into the surgical hernia patch;

FIG. 5 is a fragmentary diagrammatical cross-sectional view of the incision in the abdominal wall of FIG. 2, wherein the balloon of the surgical patch applicator is inflated to expand the surgical hernia patch;

FIG. 6 is a fragmentary diagrammatical cross-sectional view of the incision in the abdominal wall of FIG. 2, wherein a stapling cartridge housing of a surgical stapling instrument is inserted into the incision and into the surgical hernia patch;

FIG. 7 is a side elevational view of the surgical patch applicator of FIG. 4;

FIG. 8 is a side elevational view of the surgical stapling instrument of FIG. 6;

FIG. 9 is a side elevational view of another embodiment of a surgical stapling instrument in accordance with the present invention;

FIG. 10 is a side elevational view another embodiment of a surgical stapling instrument in accordance with the present invention, with the instrument in its extended position;

FIG. 11 is a side elevational view of the surgical stapling instrument of

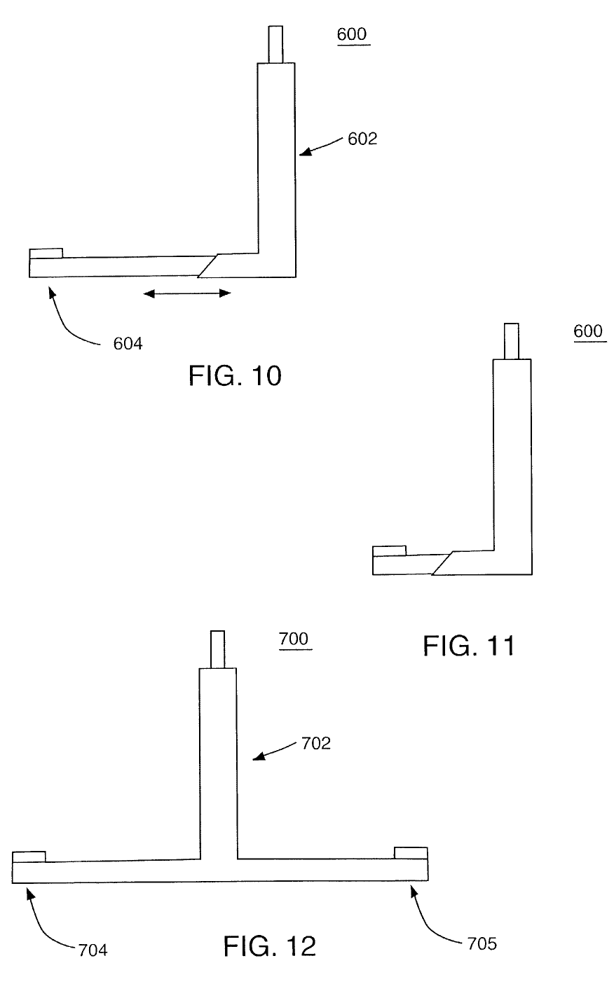

FIG. 10, with the instrument in its retracted position;

FIG. 12 is a side elevational view of another embodiment of the surgical stapling instrument having a plurality of staple cartridge housings in accordance with the preset invention;

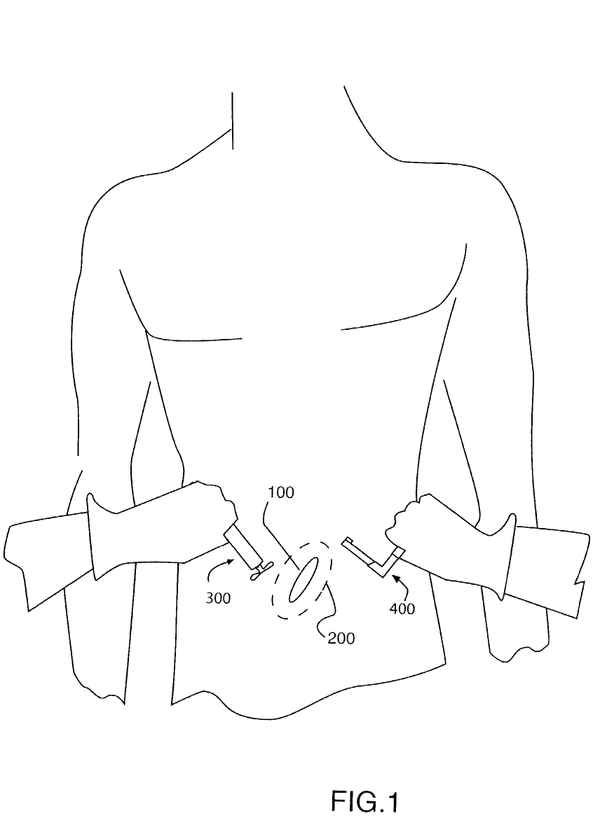

FIG. 13 is a perspective view of the surgical patch of FIG. 1; and

FIGS. 14-20 show various embodiments of surgical fasteners.

### DESCRIPTION OF PREFERRED EMBODIMENTS

Before explaining the preferred embodiments in detail, it should be noted that the invention is not limited in its application or use to the details of construction and arrangement of parts illustrated in the accompanying drawings and description, because the illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the preferred embodiments of the present invention for the convenience of the reader and are not for the purpose of limitation.

Referring now to the drawings in detail, and particularly to FIGS. 1-6, a procedure for the repair a hernia of a patient is illustrated. The surgical procedure allows a hernia to be repaired with less tension, a smaller wound or incision, superior fixation and at a potentially lower cost to the patient than traditional methods. The surgical procedure also allows a surgeon to repair a hernia more quickly than traditional techniques and without the use of sutures. Although the surgical procedure will be described in reference to a repair of an inguinal hernia, it will be recognized that the following surgical procedure can be used to repair other types of hernias and internal tissue of a patient.

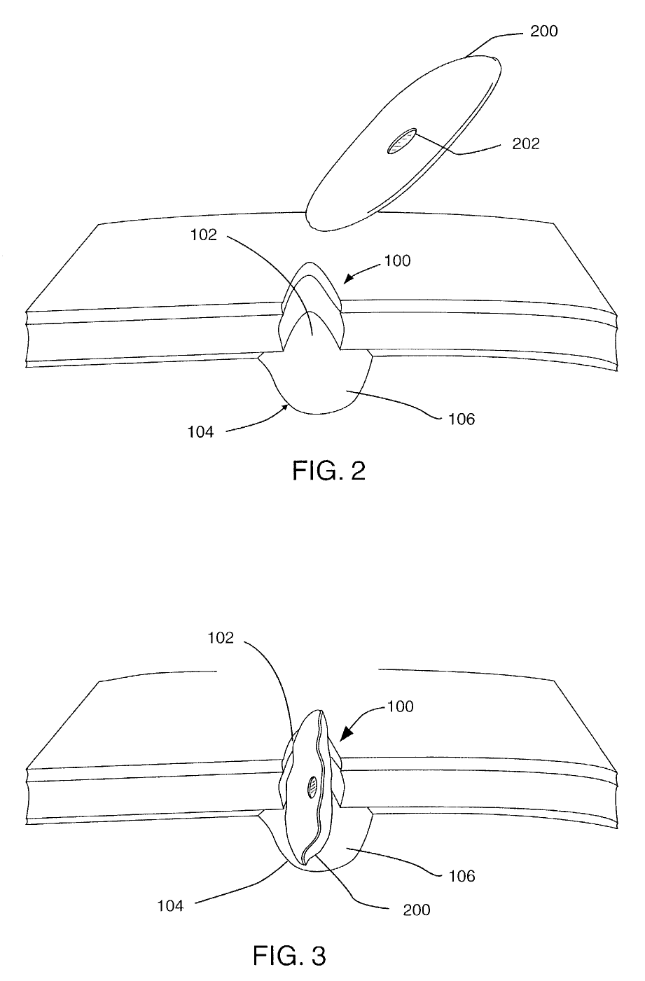

As shown in FIGS. 1-2, the surgeon creates an entrance into the patient by opening the abdominal cavity by creating a surgical incision 100 through the major abdominal muscles. The surgical incision 100 is preferably positioned approximately two to three centimeters above the location where an inguinal hernia 102 has occurred. The surgical incision 100 can be made by a blade, such as a surgical scalpel.

After the incision 100 is made in the abdominal cavity, the surgeon then works through the incision 100 and uses a muscle splitting technique to dissect deeply into the patient's preperitoneal space 104. Several layers of the abdominal wall are generally separated to reach the herniated portions and to prepare an opening for the insertion of a surgical hernia patch 200. During the separation, the surgeon identifies and frees up the hernia sac and creates a pocket 106 in the preperitoneal space 104 where the surgical hernia patch 200 can be inserted.

After the pocket 106 in the preperitoneal space 104 has been created, the surgeon selects a suitable surgical hernia patch 200 to be used for the repair of the patient's hernia 102. The selected surgical hernia patch 200 is folded and further compacted, as may be necessary, by the surgeon so that the selected surgical hernia patch 200 may be conveniently inserted through the incision 100 and down into the properitoneal space 104 as shown in FIG. 3.

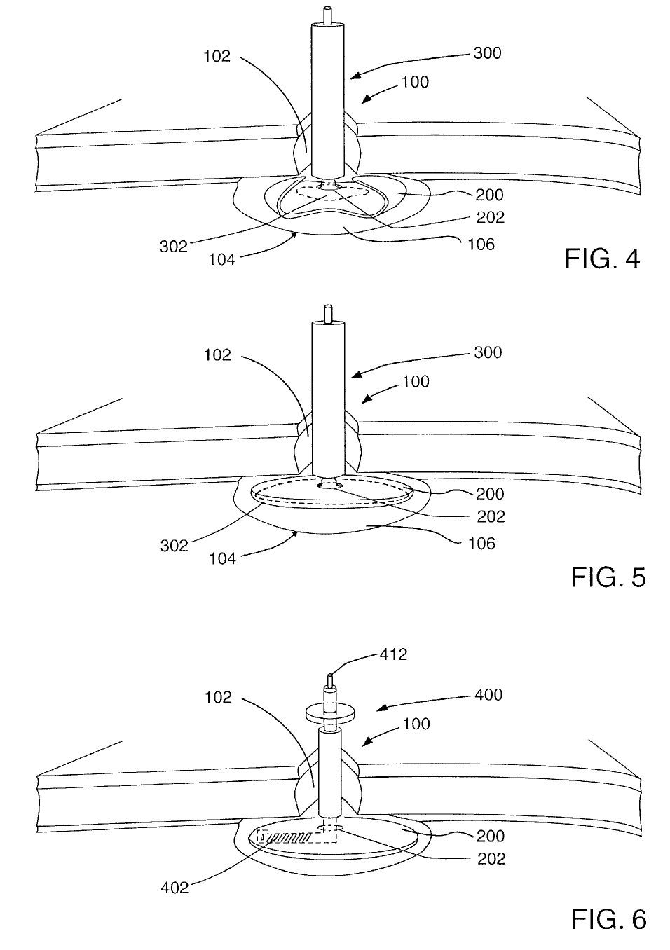

Once the surgical hernia patch 200 is inserted in the preperitoneal space 104, the surgeon can then use a hernia patch applicator 300 to conveniently and accurately position the surgical hernia patch 200 to cover the hernia 102. In order to position the patch, the surgeon inserts the hernia patch applicator 300 into the incision and into the surgical hernia patch 200 as shown in FIG. 4. A balloon 302 of the hernia patch applicator 300 is passed through a slit or hole 202 in the top layer of the surgical hernia patch 200 and into a pouch formed between the top and bottom layers of the surgical hernia patch 200. The balloon 302 is then inflated to cause the surgical hernia patch 300 to unfold and expand into a planar configuration in the pocket 106 within the preperitoneal space 104 as shown in FIG. 5, thereby causing the surgical hernia patch 200 to expand over the hernia. The hernia patch applicator 300 can easily move and expand the surgical hernia patch 200 over the hernia 102 so that the edges of the surgical hernia patch 200 overlap the circumference of the hernia 102. Once the surgical hernia patch 200 is properly positioned, the balloon 302 of the hernia patch applicator 300 is deflated and removed.

Alternatively, the surgical hernia patch 200 may initially be placed over the balloon 302 of the surgical patch applicator 300 and then inserted into the incision 100 of the patient. Thereafter, the balloon 302 may be inflated to cause the surgical hernia patch 200 to expand over the hernia 102. It will also be recognized that the surgeon may desire to use his fingers to position the surgical hernia patch 200 instead of using the hernia patch applicator 300.

Once the surgical hernia patch 200 is properly positioned, the surgeon closes the hernia 102 by applying a plurality of staples or fasteners with a surgical stapling instrument 400 to secure the surgical hernia patch 200 to the abdominal wall of the patient. In order to fasten the surgical hernia patch 200 to the abdominal wall, the surgeon inserts a staple cartridge housing 402 of the surgical stapling instrument 400 into the slit or opening 202 in the top layer of the surgical hernia patch 200 as shown in FIG. 6. When the staple cartridge housing 402 is positioned at a desired location, a staple actuator button 412 of the surgical stapling instrument 400 is pressed to drive a staple or fastener through the top layer of the surgical hernia patch 200 and into the tissue of the abdominal wall. Thereafter, the staple cartridge housing 402 is rotated to another location and the operation is repeated to drive another staple through the surgical hernia patch 200 and into the tissue. The staple cartridge housing 402 can be readily rotated to different positions to apply staples at various locations along the edges of the surgical hernia patch 200. After the surgical hernia patch 200 is secured to the patient, the surgeon removes the surgical stapling instrument 400 and closes the incision 100.

Soon after the surgery, the patient's body reacts to the surgical hernia patch 200 and scar tissue grows into the patch to permanently fix the surgical hernia patch 200 in its intended position over the repaired area, where the hernia 102 was located. The surgical hernia patch 200 also helps protect against future hernias.

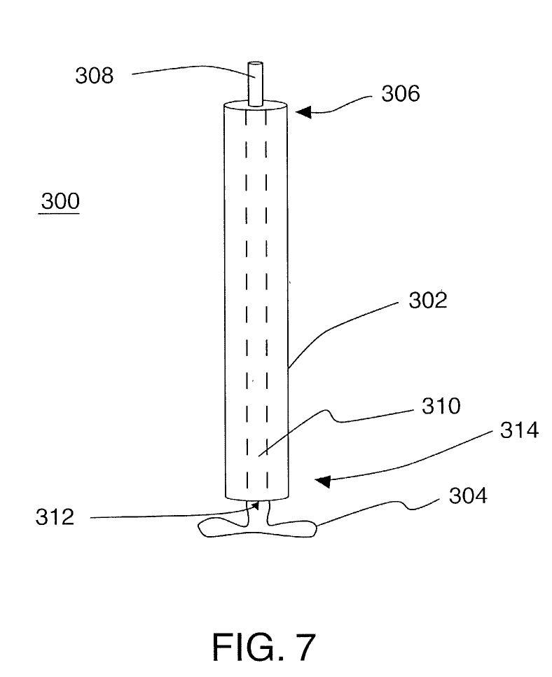

Referring now to FIG. 7, a preferred embodiment of the surgical patch applicator 300 for use in the repair of a hernia is illustrated. The surgical patch applicator 300 allows a surgical hernia patch to be readily positioned over the circumference of the hernia. The surgical patch applicator 300 preferably includes an elongated body or tube 302 and an inflatable/deflatable balloon 304.

The elongated body 302 of the surgical patch applicator 300 preferably has a substantially circular cross-section, but may have any suitable cross-section, such as a square or an elliptical cross-section. The elongated body 302 can have any suitable length depending upon the particular hernia procedure and can be constructed of any suitable material that provides sufficiently rigidity to permit insertion of the elongated body into the herniated site. The elongated body 302 can be constructed from nylon, Teflon, polyurethane, or polyethylene. It will be recognized that the elongated body 302 can be made from a variety of other materials including, for example, polypropylene, polyamide, polyethylenterephthalate, polyamide, other polymers and polycarbonates as well as other suitable forms of plastic.

The proximal end 306 of the elongated body 302 is attached to a connector or adaptor 308 through which fluid may be introduced under pressure into the balloon. The connector 308 permits the elongated body 302 to be attached or coupled to other devices, such as, a fluid source. The connector 308 can include, but is limited to, a Luer Lock connector, a quick connector, a ferrule connector, a threadable connector, and the like.

As shown in FIG. 7, the elongated body 302 of the surgical patch applicator 300 further has an interior lumen or conduit 310 positioned therein. The lumen 310 can be any suitable size and shape. The lumen 310 extends longitudinally from the proximal end 306 of the elongated body 302 to an opening or aperture 312 at the distal end 314 of the elongated body 302. The opening 312 permits the fluid to be transmitted through the lumen 310 into the interior of the balloon 304 to controllably inflate and/or deflate the balloon 304 as further described below.

The balloon 304 of the surgical patch applicator 300 is preferably attached at the distal end 314 of the elongated body 302. The balloon 304 can be made of latex, silicone rubber, polyethylene, polyamide or any other suitable material. The balloon 304 can be configured in various sizes. The balloon 304 is disposed over the opening 312 in the elongated body 302 to permit the lumen 310 to be in fluid communication with the interior of the balloon 304. As a result, when fluid is transmitted through the lumen 310 and into the interior of the balloon 304, the fluid will cause the balloon 304 to inflate. When the balloon 304 is inflated, the balloon 304 preferably expands radially outward or in a planar fashion to form a disk-like shape. FIG. 5 shows the balloon 304 in an expanded configuration in which the balloon 304 is inflated.

When the fluid is extracted or removed from the interior of the balloon 304, the balloon 304 will deflate. The fluid that may be used to inflate and deflate the balloon 304 can be a liquid, such as water or saline, or a gas, such as air, inert gas, carbon dioxide, helium, nitrogen, or the like. The fluid may be injected into and removed from the lumen 310 of the surgical patch applicator 300 by a fluid source such as, for example, a rubber bulb, a syringe, a micro pump or the like (not shown).

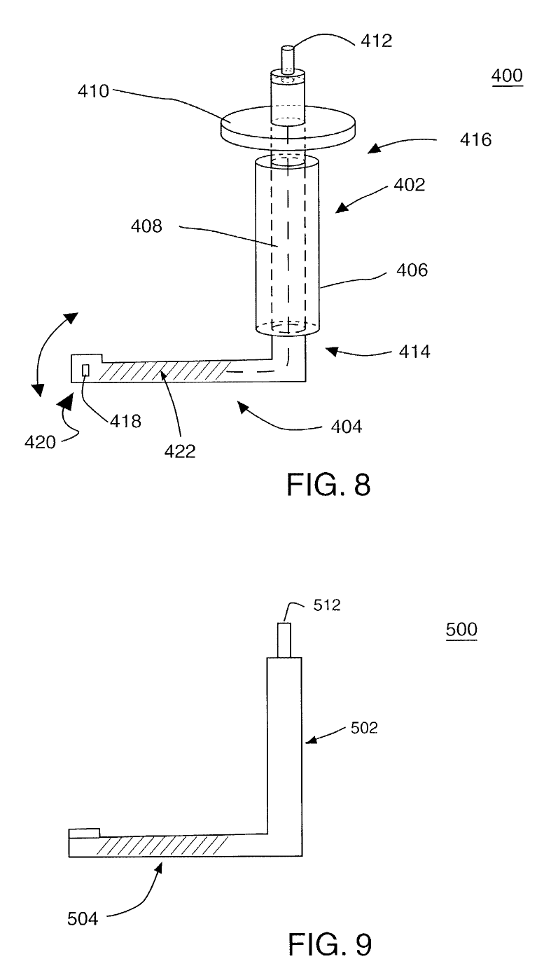

Referring now to FIG. 8, a preferred embodiment of the surgical stapling instrument 400 for attaching a surgical hernia patch to internal body tissue is illustrated. The surgical stapling instrument 400 is adapted for insertion through a slit or a slot of the surgical hernia patch in order to apply one or more surgical staples through the top layer of the surgical hernia patch and into the patient's tissue at a desired surgical site. Preferably, the surgical stapling instrument 400 applies the staples near the edges of the surgical hernia patch. The surgical stapling instrument 400 may be readily rotated to various different positions to apply the staples or fasteners at various locations along the edges of the surgical hernia patch.

As shown in FIG. 8, the surgical stapling instrument generally 400 includes a handle assembly 402 and a staple cartridge housing 404. The handle assembly 402 preferably consists of a plastic material, but may be constructed from any suitable material. The handle assembly 402 of the surgical stapling instrument 400 generally includes an outer sleeve 406, an inner sleeve or shaft 408, a rotatable control knob 410, and a stapler actuator button 412.

The outer sleeve 406 of the handle assembly 402 is substantially cylindrically shaped and is adapted to be held by a user or surgeon, but may be any suitable shape or size which allows it to be grasped by the user. The outer sleeve 406 may include a manual grip to facilitate grasping of the surgical stapling instrument 400 by a user.

As shown in FIG. 8, the distal end 414 of the inner sleeve 408 is connected to the staple cartridge housing 404, and the proximal end 416 of the inner sleeve 408 is coupled to the staple actuating button 412. The staple actuating button 412 causes the surgical stapling instrument 400 to advance and drive a staple or fastener disposed in the staple cartridge housing 404 into the tissue at the surgical site.

The rotatable control knob 410 of the surgical stapling instrument 400 is attached to inner sleeve 408 and is adapted to rotate the inner sleeve 408 about its longitudinal axis, thereby rotating the staple cartridge housing 404 relative to the outer sleeve 406 of the handle assembly 402. The control knob 410 can rotate the staple cartridge housing 404 a full 360 degrees. The control knob 410 preferably comprises a disc-like member, but may be any suitable shape or size which allows it to be rotated by the user.

The handle assembly 402 may also include a ratchet mechanism (not shown) to allow the user to set and retain the staple cartridge housing 404 at different rotational positions relative to the longitudinal axis of the outer sleeve 406 of the handle assembly 402. The ratchet mechanism may be formed by a plurality of ratchet teeth on the outer wall of the inner sleeve 408 of the handle assembly 402 for engaging a pair of notches or detents mounted on inner wall of the outer sleeve 406. The ratchet teeth and detents provide a ratchet mechanism for controlling and retaining the staple cartridge housing 404 in different rotational positions relative the longitudinal axis of the inner sleeve 408. The notches can provide a series of stop positions which correspond to angular orientations preferably of 0, 45, 90, 135, 180, 225, 270, 315, and 360 degrees relative to the longitudinal axis of the inner sleeve 408. In one embodiment, the inner wall of the outer sleeve is provided with notches which allow the staple cartridge housing 404 to be rotated in 8 equal angular increments of 45 degrees. It will be recognized that the outer wall of the inner sleeve may contain the notches while the inner wall of the outer sleeve contains the ratchet teeth.

Referring still to FIG. 8, the staple cartridge housing 404 is mounted for rotation about the longitudinal axis of the handle assembly 402. The control knob 410 of the surgical stapling instrument 400 can be turned by a user to rotate the staple cartridge housing 404 in order to adjust the rotational position of the staple cartridge housing 404 relative to the handle assembly 402. The staple cartridge housing 404 includes a staple actuating device 418 and a staple or fastener cartridge 422. The staple cartridge 422 of the surgical cartridge housing 404 contains a plurality of staplers or fasteners that can be driven upwardly relative to the longitudinal axis of the handle assembly 402 for placement in tissue. The staple actuating device 418 advances the staple and drives the staple into the tissue. The staple actuating device 418 preferably prevents more than one fastener from being placed in the “ready” position. A variety of actuation and fastener feeding mechanisms may be employed to advance the staplers or fasteners in the staple cartridge 422 of the surgical stapling instrument 400 and to place the fasteners in the tissue at the surgical site.

In use, the stapling cartridge housing 404 is positioned at the desired position over the surgical site by operating the rotatable control knob 410 to adjust the rotational orientation of the staple cartridge housing 404. With the staple cartridge housing 404 adjusted to the desired orientation, the staple actuator button 412 is squeezed to actuate the staple actuating device 418 to apply one of the staples to the tissue at the surgical site. Thereafter, the staple cartridge housing 404 is rotated to another location and the operation is repeated to apply another staple to the tissue. These features of the surgical stapling instrument 400 allow the staple cartridge housing 404 to be aligned with the desired region of the internal body tissue to which the staple or fastener is applied. Although the surgical stapling instrument 400 is described as a single load device, it will be recognized that it may be multiple load device. It will also be recognized that the surgical stapling instrument 400 may use any suitable staple or fastener, such as a surgical anchor, a surgical screw, or the like.

Referring now to FIG. 9, another embodiment of a surgical stapling instrument 500 is illustrated. The surgical stapling instrument 500 in many respects corresponds in construction and function to the previously described surgical stapling instrument 400 of FIG. 8. Components of the surgical stapling instrument 500 which generally correspond to those components to the surgical stapling instrument 400 of FIG. 8 are designated by like reference numerals in the 500 hundred series. As shown in FIG. 9, the surgical stapling instrument 500 generally includes a handle assembly 502, a staple cartridge housing 504, and a staple actuating button 512. In this embodiment, the surgeon manually rotates the handle assembly to rotate the staple cartridge housing 504. It will also be recognized that a gripping member or outer sleeve may be coupled to the handle assembly 502.

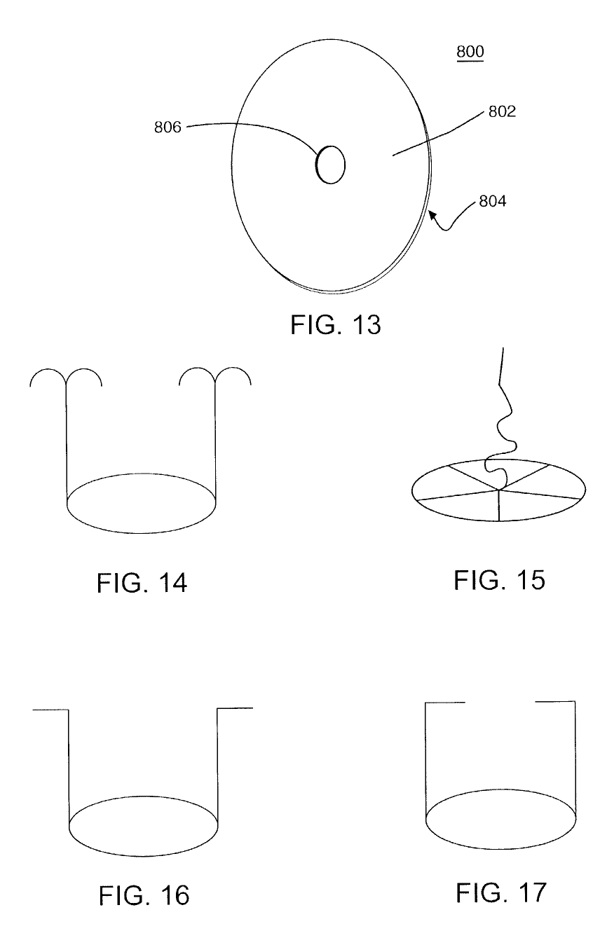

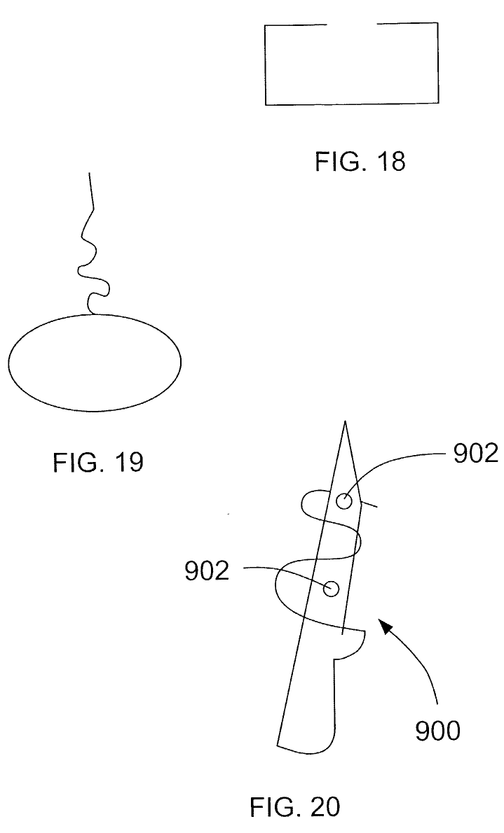

Referring now to FIGS. 10-11, another embodiment of a surgical stapling instrument 600 is illustrated. The surgical stapling instrument 600 in many respects corresponds in construction and function to the previously described surgical stapling instrument 500 of FIG. 9. Components of the surgical stapling instrument 600 which generally correspond to those components to the surgical stapling instrument 500 of FIG. 9 are designated by like reference numerals in the 600 hundred series. As shown in FIGS. 10-11, the staple cartridge housing 604 of the surgical stapling instrument 600 can be extended and retracted to facilitate the insertion into a surgical hernia patch and/or application of fasteners. FIG. 11 shows the surgical stapling instrument 600 in its retracted position while FIG. 10 shows the surgical stapling instrument in its extended position. It will be recognized that the surgical stapling instrument 600 may include an outer sleeve (not shown) to facilitate rotation of the staple cartridge housing as described in reference to FIG. 8.

Referring now to FIG. 12, another embodiment of a surgical stapling instrument 700 is illustrated. The surgical stapling instrument 700 in many respects corresponds in construction and function to the previously described surgical stapling instrument 500 of FIG. 9. Components of the surgical stapling instrument 700 which generally correspond to those components to the surgical stapling instrument 500 of FIG. 9 are designated by like reference numerals in the 700 hundred series. As shown in FIG. 12, the surgical stapling instrument 700 includes a second staple cartridge housing 705. It is contemplated that the surgical stapling instrument can have any suitable number of staple cartridge housings. The surgical stapling instrument 600 may include an outer sleeve (not shown) to facilitate rotation of the staple cartridge housing as described in reference to FIG. 8. It will be recognized that the stapling cartridge housings 704 and 705 of surgical stapling instrument 700 may be retracted and expanded as described in reference to FIGS. 10 and 11.

Referring now to FIG. 13, one embodiment of a surgical hernia patch 800 for implanting within a patient's body space for the repair a hernia is shown. The surgical hernia patch is composed of a top layer 802 and a bottom layer 804. The top and bottom layer are preferably constructed of a polypropylene material. The top and bottom layer are secured together near their outer edges to hereby form a pocket therebetween. The top layer preferably has a circular opening 806 adapted to receive a balloon of a surgical patch applicator and a staple cartridge housing of a surgical stapling instrument. Preferably, the patch does not contain memory recoil ring that is typically located near the perimeter of the patch.

FIGS. 14-19 illustrate a variety of fasteners that can be used to attach a surgical hernia patch to the tissue of the patient. The fasteners can be constructed from any suitable material. FIG. 20 shows another embodiment of a fastener 900 that can be used to attach a surgical hernia patch to the tissue of the patient. The fastener 900 can be filled with an adhesive substance, such as bio-glue, to facilitate the attachment of the fastener to the tissue. When the fastener 900 is applied to the tissue, the adhesive substance is forced out of the fastener 900 through at least one aperture or hole 902 and into the surrounding tissue.

The surgical apparatus and procedures described above allow a hernia to be repaired with less tension, a smaller wound or incision, superior fixation and at a potentially lower cost to the patient than traditional methods. The surgical procedures also allow a surgeon to repair a hernia more quickly than traditional techniques and without the use of sutures.

Although the present invention has been described in detail by way of illustration and example, it should be understood that a wide range of changes and modifications can be made to the preferred embodiments described above without departing in any way from the scope and spirit of the invention. For example, a fiber optic visualization apparatus can be incorporated into any of the surgical apparatus described above. Thus, the described embodiments are to be considered in all respects only as illustrative and not restrictive, and the scope of the invention is, therefore, indicated by the appended claims rather than the foregoing description. All changes that come within the meaning and range of equivalency of the claims are to be embraced within their scope.

What is claimed is:

1. A method for repairing a hernia using a surgical fastening device and a surgical patch, comprising the steps of:
making an incision through a skin layer of a patient near the hernia;
creating an entrance into the preperitoneal space above the peritoneum at a location above the hernia;
identifying and freeing a hernia sac;
creating a pocket in the preperitoneal space;
directing the surgical patch down through the incision and into the preperitoneal space;
expanding the surgical patch in the preperitoneal space;
inserting a distal end of the surgical fastening device through the incision and into the surgical patch;
actuating the surgical fastening device to drive a fastener through the surgical patch and into the tissue of the patient;
moving the distal end of the surgical fastening device to another location;
actuating the surgical fastening device to drive a second fastener through the surgical patch and into the tissue of the patient; and
closing the incision with stitches.

2. The method of claim 1 wherein the surgical patch is expanded by inserting a balloon in the pouch of the surgical patch and inflating the balloon.

3. The method of claim 2 wherein the balloon is inflating by transmitting a fluid through a lumen of a surgical apparatus and into the interior of the balloon.

4. The method of claim 3 wherein the fluid comprises one of a liquid and a gas.

5. The method of claim 2 further comprising the step of deflating the balloon and removing the balloon from the pouch.

6. A method of positioning a surgical patch over a hernia of a patient using a surgical device, comprising the steps of:
making an incision through a skin layer of the patient near the hernia;
inserting the surgical patch down through the incision;
inserting a balloon of the surgical device through the incision and into the surgical patch;
inflating the balloon of the surgical device to expand the surgical patch over the hernia;
deflating the balloon; and
removing the balloon from the surgical patch.

7. The method of claim 6 further comprising the steps of:
inserting a distal end of a surgical stapling apparatus into the surgical patch; and
attaching the surgical patch to the tissue with at least one fastener using the surgical stapling apparatus.

8. A surgical stapling instrument for applying one or more surgical fasteners to attach a surgical patch to internal body tissue, comprising:
a handle assembly having an outer and inner sleeve;
the inner sleeve rotatably coupled to the outer sleeve for rotational movement relative to the outer sleeve about a longitudinal axis of the inner sleeve;
a staple cartridge housing mounted to the inner sleeve and adapted to receive a plurality of staples, the staple cartridge housing dimensioned for insertion through an incision and having a staple actuator mechanism;
an actuation button operatively coupled to the staple actuator mechanism to cause the staple actuator mechanism to drive at least one staple into the tissue; and
a control knob mounted to the inner sleeve for controlling the rotation of the inner sleeve and the rotation of the staple cartridge housing relative to the outer sleeve.

9. The surgical stapling instrument of claim 8 further comprising means for retaining said staple cartridge housing at different rotational orientations relative to the outer sleeve.

10. The surgical stapling instrument of claim 9 wherein the retaining means includes at least one ratchet member and at least one detent.

11. The surgical stapling instrument of claim 10 wherein the at least one ratchet member is disposed on the inner wall of the outer sleeve and the at least one detent is disposed on the outer wall of the inner sleeve.

12. The surgical stapling instrument of claim 10 the staple actuator mechanism drives at least one staples substantially parallel to the longitudinal axis of the inner sleeve.

13. A surgical stapling instrument for applying at least one surgical staple to fasten a surgical hernia patch to internal body tissue, comprising:
a handle assembly have a longitudinal axis;
a staple cartridge housing mounted to the handle assembly and adapted to receive the at least one staple, the staple cartridge housing dimensioned for insertion through an incision and having a staple actuator mechanism for applying the at least one staple at an orientation substantially parallel to the longitudinal axis of the handle assembly; and
an actuation mechanism operatively coupled to the staple actuator mechanism to operate the staple actuation mechanism.

14. The surgical stapling instrument of claim 13 wherein the staple cartridge housing is operable between a retracted position and an extended position.

15. The surgical stapling instrument of claim 13 further comprising a second staple cartridge housing mounted to the inner sleeve and adapted to receive a plurality of staples.

16. A hernia patch applicator for positioning a surgical hernia patch over a hernia defect, the applicator comprising:
an elongated member having a first end and a second end, the second end sized for insertion into an opening of the surgical hernia patch;
a lumen extending through the elongated member; and
a balloon coupled to the second end of the elongated member, the balloon being inflatable in a planar direction to expand the surgical hernia patch over the hernia defect when a fluid is introduced into the lumen.

17. The hernia patch applicator of claim 16 further comprising a fluid source in communication with the lumen for inflating and deflating the balloon by controlled transmission of the fluid.

18. The hernia patch applicator of claim 17 wherein the fluid comprises one of a liquid and a gas.
